(19) United States
(12) Patent Application Publication
(10) Pub. No.: US 2020/0305834 A1
(43) Pub. Date: Oct. 1, 2020

(54) ULTRASOUND OBSERVATION APPARATUS AND ULTRASONIC ENDOSCOPE SYSTEM

(71) Applicant: FUJIFILM Corporation, Tokyo (JP)

(72) Inventors: Masanobu UCHIHARA, Kanagawa (JP); Masayuki TAKAHIRA, Kanagawa (JP)

(73) Assignee: FUJIFILM Corporation, Tokyo (JP)

(21) Appl. No.: 16/782,749

(22) Filed: Feb. 5, 2020

(30) Foreign Application Priority Data
Mar. 26, 2019 (JP) 2019-058862

(51) Int. Cl. A61B 8/12; A61B 8/00; A61B 1/00; A61B 8/06

(52) U.S. Cl. CPC A61B 8/12; A61B 8/06; A61B 1/00002; A61B 8/4444

(57) ABSTRACT

An ultrasonic endoscope system includes an ultrasonic endoscope and an ultrasonic endoscope apparatus, in which the ultrasound observation apparatus includes a B-mode image generator that generates, on the basis of an ultrasound signal acquired by the ultrasonic endoscope, a B-mode image obtained by converting an amplitude of the ultrasound signal into a brightness; a region-of-interest setting unit that sets a region of interest ROI in the B-mode image; a blood flow image generator that generates a blood flow image of the region of interest ROI on the basis of the ultrasound signal; and an image display part that is capable of displaying the B-mode image or a composite image of the B-mode image and the blood flow image, and the region-of-interest setting unit recognizes a procedure performed using the ultrasonic endoscope and sets the region of interest ROI in accordance with the recognized procedure.

### CROSS-REFERENCE TO RELATED APPLICATIONS

The present application claims priority under 35 U.S.C. § 119 to Japanese Patent Application No. 2019-058862, filed on Mar. 26, 2019. Each of the above application(s) is hereby expressly incorporated by reference, in its entirety, into the present application.

### BACKGROUND OF THE INVENTION

### 1. Field of the Invention

The present invention relates to an ultrasound observation apparatus and an ultrasonic endoscope system.

### 2. Description of the Related Art

In general, an ultrasound image used for examination, diagnosis, and the like in a medical field is a B-mode image obtained by imaging brightness values according to an amplitude of an ultrasound signal, but a blood flow image obtained by imaging the location, strength, direction, speed, and the like of a blood flow detected using the ultrasonic Doppler phenomenon is also used.

Compared with the B-mode image, the blood flow image needs processing and time for generating an image. For this reason, a partial region in the B-mode image is set as a region of interest (ROI), and a blood flow image is generated only for the ROI (for example, see JP2015-171425A).

In an ultrasound observation apparatus disclosed in JP2015-171425A, an ROI in a color flow mode for generating a blood flow image is set in advance by an operator, and setting information is stored in a memory. In a case where switching from the B-mode to the color flow mode is performed, the ROI is automatically set on the basis of the setting information stored in the memory.

### SUMMARY OF THE INVENTION

As a procedure of ultrasound examination, for example, pancreas observation using an ultrasonic endoscope, endoscopic ultrasound-guided fine needle aspiration (EUS-FNA), and the like, are used. The pancreas observation and the EUS-FNA may be performed in a color flow mode, but preferable ROIs in the respective procedures are different from each other. In a case where an operator resets the ROI in accordance with the procedure, an operation burden necessary for the setting becomes large.

An object of the invention is to provide an ultrasound observation apparatus and an ultrasonic endoscope system capable of easily performing setting an ROI.

According to an aspect of the present invention, there is provided an ultrasound observation apparatus comprising a B-mode image generator that generates, on the basis of an ultrasound signal acquired by an ultrasonic endoscope, a B-mode image obtained by converting an amplitude of the ultrasound signal into a brightness; a region-of-interest setting unit that sets a region of interest in the B-mode image; a blood flow image generator that generates a blood flow image of the region of interest on the basis of the ultrasound signal; and an image display part that is capable of displaying the B-mode image or a composite image of the B-mode image and the blood flow image, in which the region-of-interest setting unit recognizes a procedure performed using the ultrasonic endoscope and sets the region of interest in accordance with the recognized procedure.

Further, according to another aspect of the present invention, there is provided an ultrasonic endoscope system comprising: an ultrasonic endoscope; and the ultrasound observation apparatus.

According to the invention, it is possible to provide an ultrasound observation apparatus and an ultrasonic endoscope system capable of easily setting an ROI.

### BRIEF DESCRIPTION OF THE DRAWINGS

FIG. 1 is a schematic view showing an example of an ultrasonic endoscope system for illustrating an embodiment of the invention.

FIG. 2 is a plan view of a distal end part of an insertion part of the ultrasonic endoscope shown in FIG. 1.

FIG. 3 is a sectional view of the distal end part of the insertion part of the ultrasonic endoscope shown in FIG. 2.

FIG. 4 is a block diagram showing an ultrasound observation apparatus shown in FIG. 1.

FIG. 5 is a schematic view of an ROI setting example.

FIG. 6 is a schematic view of another ROI setting example.

### DESCRIPTION OF THE PREFERRED EMBODIMENTS

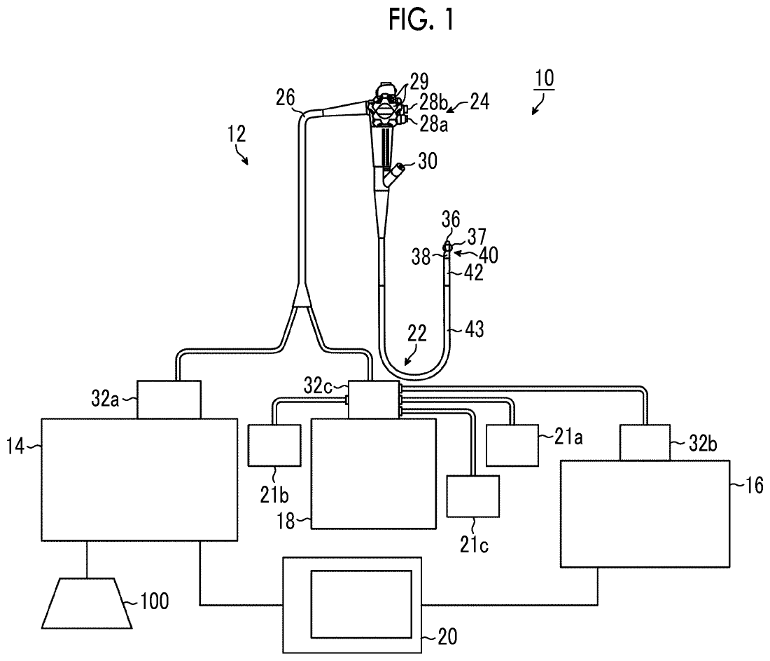

FIG. 1 shows an example of an ultrasonic endoscope system for illustrating an embodiment of the invention.

An ultrasonic endoscope system 10 is used for diagnosing a state of an observation target portion inside the body of a patient that is a subject using ultrasonic waves. Here, the observation target portion is a portion that is difficult to examine from the body surface side (outside) of the patient, such as the gallbladder or pancreas. By using the ultrasonic endoscope system 10, it is possible to make an ultrasonic diagnosis of the state of the observation target portion and the presence or absence of an abnormality through a digestive tract such as an esophagus, a stomach, and a duodenum that are body cavities of a patient.

As shown in FIG. 1, the ultrasonic endoscope system 10 includes an ultrasonic endoscope 12, an ultrasound observation apparatus 14, an endoscope processor 16, a light source device 18, a monitor 20, and a console 100. Further, as shown in FIG. 1, a water supply tank 21a, a suction pump 21b, and an air supply pump 21c are provided as accessory devices of the ultrasonic endoscope system 10. Further, a pipe line (not shown) that serves as a flow path for water and gas is formed in the ultrasonic endoscope 12.

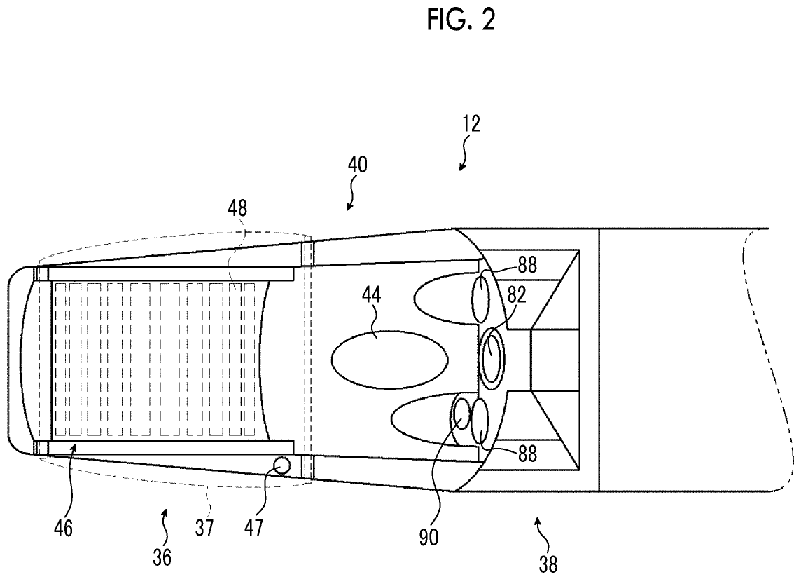

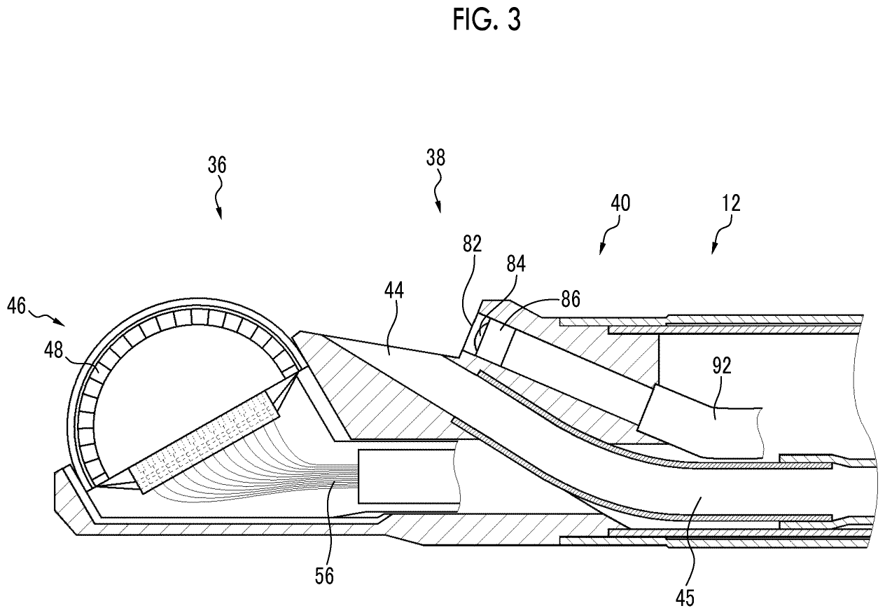

As shown in FIG. 1, the ultrasonic endoscope 12 which is an endoscope scope includes an insertion part 22 that is inserted into a body cavity of a patient, and an operation part 24 that is operated by an operator (user) such as a doctor or a technician. Further, as shown in FIGS. 2 and 3, an ultrasound vibrator unit 46 including a plurality of ultrasound vibrators 48 is attached to a distal end part 40 of the insertion part 22.

With the function of the ultrasonic endoscope 12, the operator may acquire an endoscope image of an inner wall of the body cavity of the patient and an ultrasound image of the observation target portion. The endoscope image is an image obtained by imaging the inner wall of the body cavity of the patient using an optical procedure. The ultrasound image is an image obtained by receiving reflected waves (echoes) of ultrasonic waves transmitted from the body cavity of the patient toward the observation target portion and imaging a reception signal thereof.

The ultrasound observation apparatus 14 is connected to the ultrasonic endoscope 12 through a universal cord 26 and an ultrasound connector 32a provided at an end part thereof, as shown in FIG. 1. The ultrasound observation apparatus 14 controls an ultrasound vibrator unit 46 of the ultrasonic endoscope 12 to transmit ultrasonic waves to the ultrasound vibrator unit 46. Further, the ultrasound observation apparatus 14 images a reception signal in a case where the ultrasound vibrator unit 46 receives reflected waves (echoes) of ultrasonic waves to generate an ultrasound image.

As shown in FIG. 1, the endoscope processor 16 is connected to the ultrasonic endoscope 12 through the universal cord 26 and an endoscope connector 32b provided at an end part of the universal cord 26. The endoscope processor 16 acquires image data of an observation target adjacent portion imaged by the ultrasonic endoscope 12 (specifically, a solid-state imaging element 86 to be described later), and performs predetermined image processing with respect to the acquired image data to generate an endoscope image. The observation target adjacent portion is a portion of the inner wall of the body cavity of the patient, which is adjacent to the observation target portion.

The ultrasound observation apparatus 14 and the endoscope processor 16 are configured by two apparatuses (computers) that are separately provided. However, the invention is not limited thereto, and both the ultrasound observation apparatus 14 and the endoscope processor 16 may be configured by a single apparatus.

As shown in FIG. 1, the light source device 18 is connected to the ultrasonic endoscope 12 through the universal cord 26 and a light source connector 32c provided at the end part thereof. The light source device 18 emits white light formed of three primary color lights of red light, green light, and blue light or specific wavelength light in imaging the observation target adjacent portion using the ultrasonic endoscope 12. The light emitted from the light source device 18 propagates in the ultrasonic endoscope 12 through a light guide (not shown) included in the universal cord 26, and then, is emitted from the ultrasonic endoscope 12 (specifically, an illumination window 88 to be described later). Thus, the observation target adjacent portion is illuminated by the light from the light source device 18.

As shown in FIG. 1, the monitor 20 is connected to the ultrasound observation apparatus 14 and the endoscope processor 16, and displays an ultrasound image generated by the ultrasound observation apparatus 14 and an endoscope image generated by the endoscope processor 16. Regarding the display of the ultrasound image and the endoscope image, either one of the images may be switched and displayed on the monitor 20, or both the images may be simultaneously displayed. Further, a configuration in which the display methods are able to be discretionally selected or changed may be used. In this embodiment, the ultrasound image and the endoscope image are displayed on one monitor 20, but an ultrasound image display monitor and an endoscope image display monitor may be separately provided. Further, a display form other than the monitor 20 may be used, for example, a form in which an ultrasound image and an endoscope image are displayed on a display of a personal terminal carried by an operator may be used.

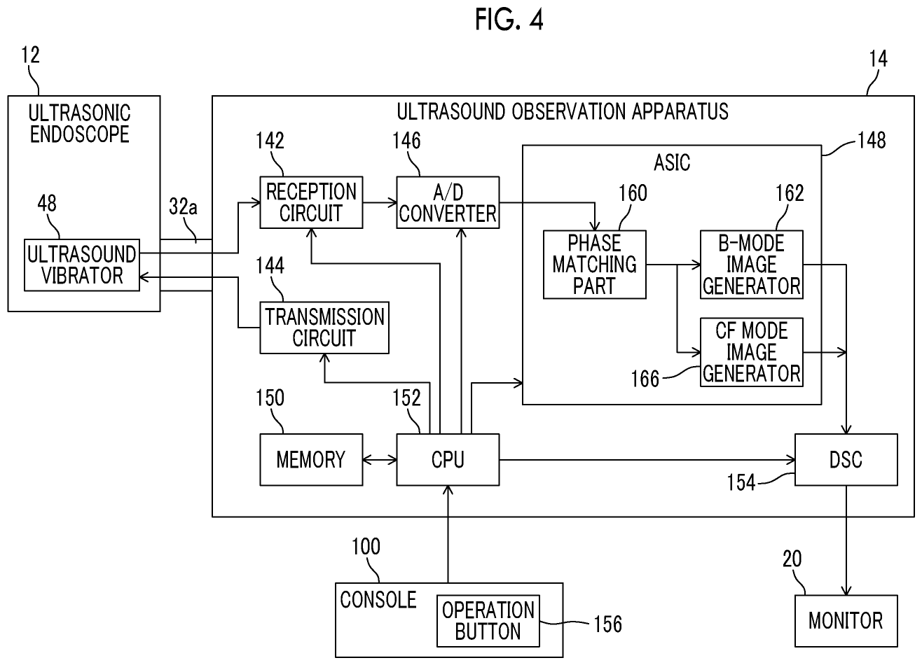

The console 100 (input part) is an input device provided for an operator to input information necessary for ultrasonic diagnosis or for an operator to instruct the ultrasound observation apparatus 14 to start the ultrasonic diagnosis. The console 100 includes, for example, a keyboard, a mouse, a trackball, a touch pad, a touch panel, and the like, and is connected to a CPU 152 of the ultrasound observation apparatus 14 as shown in FIG. 4. In a case where the console 100 is operated, the CPU 152 of the ultrasound observation apparatus 14 controls each part of the apparatus (for example, a reception circuit 142 and a transmission circuit 144 to be described later) in accordance with the operation content.

More specifically, the operator inputs examination information (for example, examination order information including a date and an order number, and patient information including a patient ID and a patient name) using the console 100 before starting the ultrasonic diagnosis. In a case where the operator instructs the start of the ultrasonic diagnosis through the console 100 after completing the input of the examination information, the CPU 152 of the ultrasound observation apparatus 14 controls respective parts of the ultrasound observation apparatus 14 so that the ultrasonic diagnosis is executed on the basis of the input examination information.

Further, the operator can set various control parameters using the console 100 in executing the ultrasonic diagnosis. Examples of the control parameters include selection of an ultrasound image generation mode. The selectable ultrasound image generation modes include a brightness (B) mode and a color flow (CF) mode, for example. The B-mode is a mode for displaying a tomographic image by converting the amplitude of an ultrasonic echo into the brightness. The CF mode is a mode in which an average blood flow velocity, a flow fluctuation, a flow signal strength, a flow power, and the like are mapped to various colors and displayed to be superimposed on a B-mode image.

Next, a configuration of the ultrasonic endoscope 12 will be described.

The ultrasonic endoscope 12 includes the insertion part 22 and the operation part 24 as shown in FIG. 1. As shown in FIG. 1, the insertion part 22 includes the distal end part 40, a bending part 42, and a flexible part 43 in order from the distal end side (free end side). As shown in FIG. 2, the distal end part 40 is provided with an ultrasound observation part 36 and an endoscope observation part 38.

Further, as shown in FIGS. 2 and 3, the distal end part 40 is provided with a treatment instrument outlet 44. The treatment instrument outlet 44 serves as an outlet of a treatment instrument (not shown) such as a pair of forceps, a puncture needle, or a high-frequency knife, and also serves as a suction port for sucking a sucked substance such as blood and filth in the body.

Further, as shown in FIG. 2, a cleaning nozzle 90 formed to clean surfaces of an observation window 82 and an illumination window 88 is provided at the distal end part 40. Air or cleaning liquid is ejected from the cleaning nozzle 90 toward the observation window 82 and the illumination window 88.

Further, as shown in FIGS. 1 and 2, a balloon 37 that is able to be inflated and deflated is attached to the distal end part 40 at a position where the ultrasound vibrator unit 46 is covered. The balloon 37 is disposed in the body cavity of the patient together with the ultrasound vibrator unit 46. Then, water (specifically, de-aired water) as an ultrasonic transmission medium is injected into the balloon 37 from a water supply port 47 formed in the vicinity of the ultrasound vibrator unit 46 at the distal end part 40, and thus, the balloon 37 is inflated. In a case where the inflated balloon 37 comes into contact with the inner wall of the body cavity (for example, around the observation target adjacent portion), air is excluded from between the ultrasound vibrator unit 46 and the inner wall of the body cavity. Thus, it is possible to prevent attenuation of ultrasonic waves and their reflected waves (echoes) in the air.

As shown in FIG. 1, the bending part 42 is a part provided on a proximal end side (a side opposite to the side where the ultrasound vibrator unit 46 is provided) with reference to the distal end part 40 in the insertion part 22, which is able to be freely bent. As shown in FIG. 1, the flexible part 43 is a part that connects the bending part 42 and the operation part 24, has flexibility, and is provided in an elongated state.

As shown in FIG. 1, the operation part 24 is provided with a pair of angle knobs 29 and a treatment instrument insertion port 30. In a case where each angle knob 29 is rotationally moved, the bending part 42 is remotely operated to be bent and deformed. By this deformation operation, the distal end part 40 of the insertion part 22 provided with the ultrasound observation part 36 and the endoscope observation part 38 may be directed in a desired direction. The treatment instrument insertion port 30 is a hole formed for insertion of a treatment instrument such as a pair of forceps, and communicates with the treatment instrument outlet 44 through a treatment instrument channel 45 (see FIG. 3).

As shown in FIG. 1, the operation part 24 is provided with an air/water supply button 28a for opening or closing an air/water supply pipe line (not shown) that extends from a water supply tank 21a, and a suction button 28b for opening or closing a suction line (not shown) that extends from a suction pump 21b. A gas such as air sent from an air supply pump 21c and water in the water supply tank 21a flow through the air/water supply pipe line. In a case where the air/water supply button 28a is operated, a part to be opened of the air/water supply pipe line is switched, and gas and water ejection ports are also switched in a corresponding form between the cleaning nozzle 90 and the water supply port 47. That is, through the operation of the air/water supply button 28a, the cleaning of the endoscope observation part 38 and the inflation of the balloon 37 may be selectively performed.

The suction line is provided for sucking a sucked substance in the body cavity sucked from the cleaning nozzle 90 or for sucking the water in the balloon 37 through the water supply port 47. In a case where the suction button 28b is operated, a portion to be opened of the suction line is switched, and the suction port is also switched in a corresponding form between the cleaning nozzle 90 and the water supply port 47. That is, an object sucked by the suction pump 21b may be switched through the operation of the suction button 28b.

As shown in FIG. 1, at the other end part of the universal cord 26, the ultrasound connector 32a connected to the ultrasound observation apparatus 14, the endoscope connector 32b connected to the endoscope processor 16, and the light source connector 32c connected to the light source device 18 are provided. The ultrasonic endoscope 12 is detachably connected to the ultrasound observation apparatus 14, the endoscope processor 16, and the light source device 18 through the connectors 32a, 32b, and 32c, respectively.

Next, among the components of the ultrasonic endoscope 12, the ultrasound observation part 36 and the endoscope observation part 38 will be described in detail.

Ultrasound Observation Part

The ultrasound observation part 36 is a part provided for acquiring an ultrasound image, and is disposed on the distal end side in the distal end part 40 of the insertion part 22 as shown in FIGS. 2 and 3. As shown in FIG. 3, the ultrasound observation part 36 includes the ultrasound vibrator unit 46, a plurality of coaxial cables 56, and a flexible printed circuit (FPC) 60.

The ultrasound vibrator unit 46 corresponds to an ultrasonic probe (probe), and transmits and receives ultrasonic waves in the body cavity of the patient (inside the subject). More specifically, the ultrasound vibrator unit 46 transmits and receives the ultrasonic waves as a drive target vibrator among a plurality of ultrasound vibrators 48 is driven inside the body cavity of the patient. The drive target vibrator is an ultrasound vibrator 48 that is actually driven (vibrated) to emit ultrasonic waves at the time of ultrasonic diagnosis and outputs a reception signal that is an electric signal in a case where reflected waves (echo) are received.

As shown in FIG. 3, the ultrasound vibrator unit 46 according to this embodiment is a convex probe in which the plurality of ultrasound vibrators 48 are arranged in an arc shape, and transmits ultrasonic waves in a radial shape (arc shape). However, the type (model) of the ultrasound vibrator unit 46 is not particularly limited, and may be any other type that can transmit and receive ultrasonic waves, for example, a sector type, a linear type, a radial type, and the like.

Each ultrasound vibrator 48 is supplied with a pulsed drive voltage as an input signal from the ultrasound observation apparatus 14 through the coaxial cable 56. In a case where the drive voltage is applied to electrodes of the ultrasound vibrator 48, the piezoelectric element expands and contracts, so that the ultrasound vibrator 48 is driven (vibrated). As a result, pulsed ultrasonic waves are output from the ultrasound vibrator 48. Here, the amplitude of the ultrasonic waves output from the ultrasound vibrator 48 has a magnitude corresponding to the strength (output strength) in a case where the ultrasound vibrator 48 outputs the ultrasonic waves. Here, the output strength is defined as the magnitude of sound pressure of the ultrasonic waves output from the ultrasound vibrator 48.

In addition, in a case each ultrasound vibrator 48 receives reflected waves (echo) of the ultrasonic waves, the ultrasound vibrator 48 is accordingly vibrated (driven), and the piezoelectric element of each ultrasound vibrator 48 generates an electric signal. The electric signal is output from each ultrasound vibrator 48 toward the ultrasound observation apparatus 14 as an ultrasonic reception signal. Here, the magnitude (voltage value) of the electric signal output from the ultrasound vibrator 48 has a size corresponding to a reception sensitivity in a case where the ultrasound vibrator 48 receives the ultrasonic waves. Here, the reception sensitivity is defined as a ratio of the amplitude of the electric signal obtained by receiving and outputting the ultrasonic waves by the ultrasound vibrator 48 to the amplitude of the ultrasonic waves transmitted by the ultrasound vibrator 48.

Endoscope Observation Part

The endoscope observation part 38 is a part provided for acquiring an endoscope image, and is disposed on a base end side with reference to the ultrasound observation part 36, in the distal end part 40 of the insertion part 22, as shown in FIGS. 2 and 3. As shown in FIGS. 2 and 3, the endoscope observation part 38 includes the observation window 82, an objective lens 84, the solid-state imaging element 86, the illumination window 88, the cleaning nozzle 90, a wiring cable 92, and the like.

As shown in FIG. 3, the observation window 82 is provided in a state of being inclined with respect to the axial direction (longitudinal axis direction of the insertion part 22), in the distal end part 40 of the insertion part 22. Light that is incident through the observation window 82 and is reflected by the observation target adjacent portion is imaged on an imaging surface of the solid-state imaging element 86 by the objective lens 84.

The solid-state imaging element 86 photoelectrically converts reflected light from the observation target adjacent portion that has passed through the observation window 82 and the objective lens 84 and is imaged on the imaging surface, and outputs an imaging signal. As the solid-state imaging element 86, a charge coupled device (CCD), a complementary metal oxide semiconductor (CMOS), or the like may be used. A captured image signal output by the solid-state imaging element 86 is transmitted to the endoscope processor 16 by the universal cord 26 through the wiring cable 92 that elongates from the insertion part 22 to the operation part 24.

As shown in FIG. 2, the illumination window 88 is provided on both arms of the observation window 82. An emission end of a light guide (not shown) is connected to the illumination window 88. The light guide elongates from the insertion part 22 to the operation part 24, and an incident end thereof is connected to the light source device 18 connected through the universal cord 26. Illumination light emitted from the light source device 18 travels through the light guide, and is irradiated from the illumination window 88 toward the observation target adjacent portion.

Next, a configuration of the ultrasound observation apparatus 14 will be described.

The ultrasound observation apparatus 14 causes the ultrasound vibrator unit 46 to transmit and receive ultrasonic waves, and generates an ultrasound image by imaging a reception signal output from the drive target element in receiving the ultrasonic waves. Further, the ultrasound observation apparatus 14 displays the generated ultrasound image on the monitor 20.

As shown in FIG. 4, the ultrasound observation apparatus 14 includes a reception circuit 142, a transmission circuit 144, an A/D converter 146, an application specific integrated circuit (ASIC) 148, a memory 150, a central processing unit (CPU) 152, and a digital scan converter (DSC) 154.

The reception circuit 142 and the transmission circuit 144 are electrically connected to the ultrasound vibrator unit 46 of the ultrasonic endoscope 12, as shown in FIG. 4. The transmission circuit 144 configures a drive voltage supply unit, which is a circuit that supplies a drive voltage for transmitting ultrasonic waves to the drive target vibrator in order to transmit ultrasonic waves from the ultrasound vibrator unit 46. The drive voltage is a pulsed voltage signal, and is applied to electrodes of the drive target vibrator through the universal cord 26 and the coaxial cable 56.

The reception circuit 142 is a circuit that receives an electric signal output from the drive target vibrator that has received ultrasonic waves (echo), that is, a reception signal. Further, the reception circuit 142 amplifies the reception signal received from the ultrasound vibrator 48 in accordance with a control signal transmitted from the CPU 152, and delivers the amplified signal to the A/D converter 146. As shown in FIG. 4, the A/D converter 146 is connected to the reception circuit 142, converts a reception signal received from the reception circuit 142 from an analog signal to a digital signal, and outputs the converted digital signal to the ASIC 148.

As shown in FIG. 4, the ASIC 148 configures a phase matching part 160, a B-mode image generator 162, and a CF mode image generator 166. The phase matching part 160, the B-mode image generator 162, and the CF mode image generator 166 are realized by a hardware circuit such as the ASIC 148, but the invention is not limited thereto. The functions may be realized by cooperation of a central processing unit (CPU) and software (computer program) for executing a variety of data processing.

The phase matching part 160 executes a process of adding a delay time to a reception signal (received data) that is converted into a digital signal by the A/D converter 146 to perform phasing addition (addition after matching phases of the received data). A sound signal in which a focus of ultrasonic echo is narrowed down is generated by the phasing addition process.

The B-mode image generator 162 and the CF mode image generator 166 generate an ultrasound image on the basis of an electric signal output from the drive target vibrator among the plurality of ultrasound vibrators 48 in a case where the ultrasound vibrator unit 46 receives ultrasonic waves (strictly speaking, an sound signal generated by performing phasing addition with respect to received data).

The B-mode image generator 162 generates a B-mode image that is a tomographic image inside a patient (inside the body cavity). The B-mode image generator 162 corrects attenuation caused by a propagation distance according to the depth of a reflection position of ultrasonic waves by sensitivity time gain control (STC) with respect to sound signals that are sequentially generated. Further, the B-mode image generator 162 performs envelope detection processing and log (logarithmic) compression processing with respect to the corrected sound signals to generate a B-mode image (image signal).

The CF mode image generator 166 generates a blood flow image indicating blood flow information in a predetermined direction. The CF mode image generator 166 obtains an autocorrelation of a plurality of sound signals in the same direction among the sound signals that are sequentially generated by the phase matching part 160, to thereby generate a blood flow image (image signal) indicating information related to a blood flow. Then, the CF mode image generator 166 generates a CF mode image (image signal) as a color image on which the information related to the blood flow is superimposed by synthesizing the blood flow image with the B-mode image.

The DSC 154 that functions as an image display part is connected to the ASIC 148, and converts an image signal generated by the B-mode image generator 162 or the CF mode image generator 166 into an image signal in accordance with a normal television signal scanning method (raster conversion), performs a variety of necessary image processing such as gradation processing on the image signal, and then outputs the image signal to the monitor 20.

The CPU 152 functions as a controller that controls each part of the ultrasound observation apparatus 14, and as illustrated in FIG. 4, is connected to the reception circuit 142, the transmission circuit 144, the A/D converter 146, the ASIC 148, and the DSC 154 to control these devices. Specifically, as shown in FIG. 4, the CPU 152 is connected to the console 100, and at the time of ultrasonic diagnosis, controls each part of the ultrasound observation apparatus 14 in accordance with examination information and control parameters input through the console 100. Thus, an ultrasound image corresponding to the ultrasound image generation mode designated by an operator is displayed on the monitor 20.

The blood flow image generated by the CF mode image generator (blood flow image generator) 166 is generated only for an ROI in the B-mode image. The CPU 152 also functions as a region-of-interest setting unit that sets the ROI in the B-mode image, recognizes the procedure to be performed using the ultrasonic endoscope 12, and sets the ROI according to the recognized procedure. The memory (storage unit) 150 stores ROI setting information for each procedure, and the CPU 152 sets the ROI on the basis of setting information corresponding to the recognized procedure among the setting information stored in the memory 150.

The operator may adjust the position and size of the ROI set by the CPU 152 using a keyboard or the like of the console 100. In a case where the ROI set by the CPU 152 is adjusted by the operator, the setting information stored in the memory 150 is preferably updated on the basis of the adjusted ROI. Thus, thereafter, the ROI set by the CPU 152 for the same procedure reflects a preference of the operator.

Next, a method for recognizing the procedure performed by the CPU 152 will be described.

As one of the procedures performed using the ultrasonic endoscope 12, there is endoscopic ultrasound-guided fine needle aspiration (EUS-FNA). A puncture needle protrudes from the treatment instrument outlet 44 (see FIGS. 2 and 3) of the ultrasonic endoscope 12, and travels along a predetermined trajectory with respect to the ultrasound vibrator unit 46. In the EUS-FNA, a puncture guideline indicating a puncture trajectory is usually displayed to be superimposed on a B-mode image, and it is confirmed in advance whether or not there is an obstruction on the puncture trajectory.

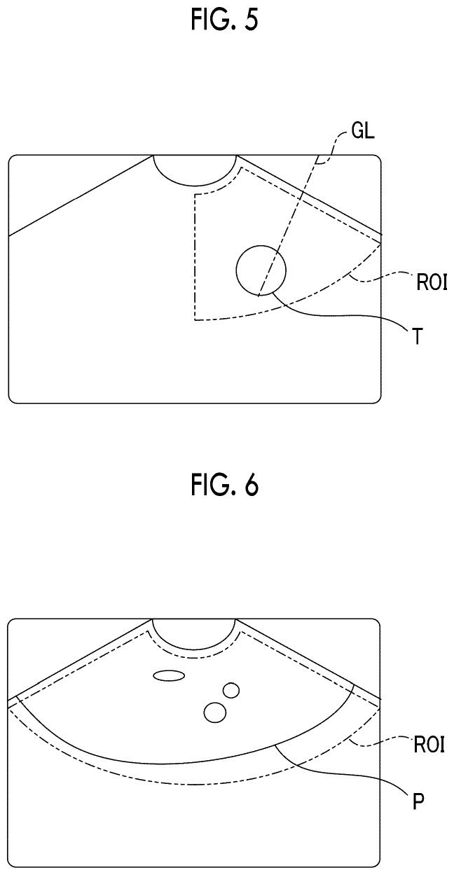

As shown in FIG. 4, the console 100 is provided with an operation button 156 for receiving an operation for displaying the puncture guideline. In a case where the operation button 156 is pressed, as shown in FIG. 5, a puncture guideline GL is displayed to be superimposed on the B-mode image. Further, in a case where the operation button 156 is pressed, the CPU 152 recognizes that the procedure is the EUS-FNA, and sets an ROI corresponding to the EUS-FNA. The puncture guideline GL is typically located in an upper right area of the B-mode image, and therefore, the ROI corresponding to the EUS-FNA is set in the upper right area of the B-mode image so as to include the puncture guideline GL. Here, a range of 50% from the right end to the center is set in a scan direction, and a range of 50% from a position directly below the ultrasound vibrator unit to the center is set in a depth direction. A symbol T indicates a puncture target.

Further, organ observation is also one of the procedures performed using the ultrasonic endoscope 12. Examples of organs observed from the stomach include the liver, pancreatic body, pancreatic tail, pancreatic head, and the gallbladder. Further, examples of the organ observed from the duodenal bulb may include the common bile duct and the gallbladder, and examples of the organ observed from the descending portion of the duodenum may include the pancreatic uncinate process and the nipple. The CPU 152 detects an organ to be observed on the basis of the B-mode image, and sets an ROI corresponding to the detected organ. The detection of the organ may be performed using a learned model, and the learned model is a model learned using a data set including a plurality of B-mode images obtained by ultrasound observation of the above organ.

FIG. 6 schematically shows a B-mode image in the case of pancreas observation, and the CPU 152 applies a learned model to the B-mode image to detect that the B-mode image is a B-mode image of the pancreas P. Further, the CPU 152 sets an ROI corresponding to the pancreas observation. Here, a range of 100% from the right end to the left end is set in the scan direction, and a range of 50% from a position directly below of the ultrasound vibrator unit to the center is set in the depth direction. The CPU 152 may adjust the position and size of the ROI so that the detected pancreas P is included in the ROI.

As described above, the CPU 152 recognizes the procedure to be performed using the ultrasonic endoscope 12, and automatically sets the ROI in accordance with the recognized procedure, thereby reducing an operation burden of the operator.

In the above-described example, the function as the controller that controls the respective parts of the ultrasound observation apparatus 14 and the function as the region-of-interest setting unit that sets an ROI are realized by one CPU 152, but may be realized by a plurality of hardware (processors) that are different for each function. Further, the function as the region-of-interest setting unit may be realized by a plurality of hardware (processors). For example, the function of detecting an observation portion on the basis of a B-mode image and the function of setting an ROI corresponding to the detected observation portion may be realized by different hardware (processors). For example, in a case where the function of detecting the observation portion on the basis of the B-mode image is realized by hardware (processor) that is different from the CPU 152, the hardware (processor) may be configured as an external module, and may be connected to the CPU 152 through a network or the like.

### EXPLANATION OF REFERENCES

10: ultrasonic endoscope system
12: ultrasonic endoscope
14: ultrasound observation apparatus
16: endoscope processor
18: light source device
20: monitor
21a: water supply tank
21b: suction pump
21c: air supply pump
22: insertion part
24: operation part
26: universal cord
28a: air/water supply button
28b: suction button
29: angle knob
30: treatment instrument insertion port
32a: ultrasound connector
32b: endoscope connector
32c: light source connector
36: ultrasound observation part
37: balloon
38: endoscope observation part
40: distal end part
42: bending part
43: flexible part
44: treatment instrument outlet
45: treatment instrument channel
46: ultrasound vibrator unit
47: water supply port
48: ultrasound vibrator
56: coaxial cable
82: observation window
84: objective lens
86: solid-state imaging element
88: illumination window
90: cleaning nozzle
92: wiring cable
100: console (input part)
142: reception circuit
144: transmission circuit
146: A/D converter
148: ASIC
150: memory (storage unit)
152: CPU (region-of-interest setting unit)
154: DSC (image display part)
156: operation button
160: phase matching part
162: B-mode image generator
166: CF mode image generator (blood flow image generator)
GL: puncture guideline
ROI: region of interest
T: puncture target
P: pancreas

What is claimed is:

1. An ultrasound observation apparatus comprising:
a B-mode image generator that generates, on the basis of an ultrasound signal acquired by an ultrasonic endoscope, a B-mode image obtained by converting an amplitude of the ultrasound signal into a brightness;
a region-of-interest setting unit that sets a region of interest in the B-mode image;
a blood flow image generator that generates a blood flow image of the region of interest on the basis of the ultrasound signal; and
an image display part that is capable of displaying the B-mode image or a composite image of the B-mode image and the blood flow image,
wherein the region-of-interest setting unit recognizes a procedure performed using the ultrasonic endoscope and sets the region of interest in accordance with the recognized procedure.

2. The ultrasound observation apparatus according to claim 1, further comprising:
an input part that receives a guideline display operation for displaying a guideline indicating a puncture trajectory of a puncture needle protruding from the ultrasonic endoscope on the image display part,
wherein the region-of-interest setting unit sets a predetermined region of interest corresponding to endoscopic ultrasound-guided fine needle aspiration in a case where the input part receives the guideline display operation.

3. The ultrasound observation apparatus according to claim 1,
wherein the region-of-interest setting unit detects an observation portion on the basis of the B-mode image, and sets a predetermined region of interest corresponding to the detected observation portion.

4. The ultrasound observation apparatus according to claim 1, further comprising:
a storage unit that stores setting information of the region of interest for each procedure,
wherein the region-of-interest setting unit sets the region of interest on the basis of the setting information corresponding to the recognized procedure among the setting information stored in the storage unit.

5. The ultrasound observation apparatus according to claim 2, further comprising:
a storage unit that stores setting information of the region of interest for each procedure,
wherein the region-of-interest setting unit sets the region of interest on the basis of the setting information corresponding to the recognized procedure among the setting information stored in the storage unit.

6. The ultrasound observation apparatus according to claim 3, further comprising:
a storage unit that stores setting information of the region of interest for each procedure,
wherein the region-of-interest setting unit sets the region of interest on the basis of the setting information corresponding to the recognized procedure among the setting information stored in the storage unit.

7. The ultrasound observation apparatus according to claim 4,
wherein in a case where the region of interest set by the region-of-interest setting unit is adjusted by a user, the storage unit updates the setting information of the region of interest on the basis of the adjusted region of interest.

8. The ultrasound observation apparatus according to claim 5,
wherein in a case where the region of interest set by the region-of-interest setting unit is adjusted by a user, the storage unit updates the setting information of the region of interest on the basis of the adjusted region of interest.

9. The ultrasound observation apparatus according to claim 6,
wherein in a case where the region of interest set by the region-of-interest setting unit is adjusted by a user, the storage unit updates the setting information of the region of interest on the basis of the adjusted region of interest.

10. An ultrasonic endoscope system comprising:
an ultrasonic endoscope; and
the ultrasound observation apparatus according to claim 1.

11. An ultrasonic endoscope system comprising:
an ultrasonic endoscope; and
the ultrasound observation apparatus according to claim 2.

12. An ultrasonic endoscope system comprising:
an ultrasonic endoscope; and
the ultrasound observation apparatus according to claim 3.

13. An ultrasonic endoscope system comprising:
an ultrasonic endoscope; and
the ultrasound observation apparatus according to claim 4.

14. An ultrasonic endoscope system comprising:
an ultrasonic endoscope; and
the ultrasound observation apparatus according to claim 5.

15. An ultrasonic endoscope system comprising:
an ultrasonic endoscope; and
the ultrasound observation apparatus according to claim 6.

16. An ultrasonic endoscope system comprising:
an ultrasonic endoscope; and
the ultrasound observation apparatus according to claim 7.

17. An ultrasonic endoscope system comprising:
an ultrasonic endoscope; and
the ultrasound observation apparatus according to claim 8.

18. An ultrasonic endoscope system comprising:
an ultrasonic endoscope; and
the ultrasound observation apparatus according to claim 9.

19. An ultrasound observation apparatus comprising:
a processor or an electric circuit configured to,
generate, on the basis of an ultrasound signal acquired by an ultrasonic endoscope, a B-mode image obtained by converting an amplitude of the ultrasound signal into a brightness;
set a region of interest in the B-mode image;
generate a blood flow image of the region of interest on the basis of the ultrasound signal; and
be capable of displaying the B-mode image or a composite image of the B-mode image and the blood flow image,
wherein the processor or the electric circuit recognizes a procedure performed using the ultrasonic endoscope and sets the region of interest in accordance with the recognized procedure.
